(19) United States
(12) Patent Application Publication
(10) Pub. No.: US 2026/0207771 A1
(43) Pub. Date: Jul. 23, 2026

(54) VIRAL VECTORS WITH ENGINEERED FUNCTION AND METHODS OF PRODUCTION THEREOF

(71) Applicant: Lilium Therapeutics Inc., Cambridge, MA (US)

(72) Inventor: Jonathan Lipsitz, Cambridge, MA (US)

(73) Assignee: Lilium Therapeutics Inc., Cambridge, MA (US)

(21) Appl. No.: 19/138,712

(22) PCT Filed: Dec. 15, 2023
(86) PCT No.: PCT/US2023/084197

§ 371 Date: Jun. 13, 2025

Related U.S. Application Data
(60) Provisional application No. 63/433,148, filed on Dec. 16, 2022.

(51) Int. Cl. A61K 47/69; A61K 31/7088; A61K 47/54; A61K 47/60; A61K 47/62; A61K 47/68; C12N 15/86

(52) U.S. Cl. CPC A61K 47/6901; A61K 31/7088; A61K 47/545; A61K 47/549; A61K 47/60; A61K 47/62; A61K 47/6849; C12N 15/86; C12N 2740/15043; C12N 2740/15045; C12N 2740/15051

(57) ABSTRACT

Provided herein are viral vectors with engineered function and methods of producing the same.

### CROSS-REFERENCE TO RELATED APPLICATIONS

This application claims priority to U.S. Provisional Patent Application Ser. No. 63/433,148, filed Dec. 16, 2022; the entire contents of which are herein incorporated by reference.

### TECHNICAL FIELD

This invention relates to the field of molecular biology and lentiviral vectors.

### BACKGROUND

Lentivirus (LV), is a genus of the Retroviridae family, which includes human immunodeficiency virus (HIV). LV is an enveloped virus with a single strand RNA of approximately 8 kilobases (kp) in length. Recent studies suggest that lentiviral vectors (LVV) may be a preferred vehicle for delivery of genes for therapeutic purposes due to its capability for long-term gene expression; specific targeting of cells, tissues, and organs; and safety profile. To date, several iterations of LVV based on modifications to HIV have been used for human clinical trials as both therapeutics and components of therapeutics in medicines. These have included chimeric antigen receptor T-cell therapies for blood cancers, and hematopoetic stem cell therapies for beta-thallassemia, cerebral adrenoleukodystrophy, and X-linked severe combined immunodeficiency. Due to LVV assembly and production limitations, it has been a technical challenge to produce LVV with novel, advanced functions including the ability to target desired cells directly in vivo while maintaining high enough delivery efficiency for therapeutic use.

### SUMMARY

The inventions described herein are based, at least in part, on the discovery that production of LVV and other enveloped vectors with the required functions for in vivo therapies is limited by the very low functional titres that result from the current processes used to produce LVV with this next-generation functionality. In fact, it has been discovered that as LVV compositions advance to add additional therapeutically relevant functions to the surface through exogenous surface proteins, there is a tradeoff between this advanced functionality and the titre at which the resulting LVV can be produced. Low titres lead to low delivery efficiencies, low potencies, and limited therapeutic applications. This is hypothesized to be due to bottlenecks in the localization of one or more desired exogenous functional proteins to the location where the vector buds off of the producer cell. Today, the entire lentiviral vector field produces vectors with exogenous surface proteins through transcription and translation of plasmids or integrated gene sequences that code for those surface proteins in the producer cell. However, this results in viral vector preparations with low titres of vectors having the one or more desired exogenous targeting surface proteins, and these low titres are insufficient for wide therapeutic use.

Increasing titer of lentiviral vectors is an active area of inquiry to improve therapeutic access and cost; however, the field today is focused on bioprocess optimizations such as changing pH, dissolved oxygen levels, mixing attributes, and concentrations. These activities result from a hypothesis that the optimum process conditions could lead to major improvements in yields: a hypothesis that has thus far not proven to be true. Herein, we have taken an entirely different approach to improve the process by fundamentally changing how LVVs are created. The industry today generates these advanced function viral vectors in a one-step production method, where genetic instructions are delivered to a producer cell to generate viral vectors with the desired exogenous functional proteins on the vector surface. The present invention is based on the discovery that an alternative production method of assembling the viral vector core first in a cell-based process, and subsequently adding the exogenous functional proteins onto the viral vector core using a cell-free process, which in turn should lead to significantly improved production yields of these advanced function viral vectors. These improvements enable an “assembly line” production method for making viral vectors one step at a time, which provides for engineering production efficiencies not present in the classical one-step cell based production methods used in the field today. The magnitude of these production improvements facilitates application of viral vector gene therapies in therapeutic areas previously inaccessible to this modality due to production constraints. The present invention is based on the discovery that chemical conjugation can be used to successfully present one or more desired exogenous surface proteins on the viral vector core after it buds off of the producer cell, thus improving the yield and increasing the titres of therapeutically effective viral vectors having one or more desired exogenous surface proteins by several orders of magnitude as compared to methods of producing viral vectors by introducing gene sequences into a producer cell alone.

Previously, the field could only make vectors that had very rudimentary targeting functions chemically conjugated to the vector, such as targeting endowed by basic peptides. Using the presently provided methods, full-length exogenous targeting proteins can be successfully added to lentivirus vectors and provide for viral vectors having much more precise targeting. Suprisingly, it has been discovered that the right combination of a viral vector core production process, conjugation technology, and combination of exogenous targeting proteins and glycoproteins provide for lentiviral vectors having improved assembly and delivery efficiency.

In view of these discoveries, provided herein are viral vectors that have improved functionality, and methods of producing the same.

Provided herein is a viral vector core having on its surface a first chemical handle. In some embodiments of any of the viral vector cores described herein, the first chemical handle comprises an azido sugar. In some embodiments of any of the viral vectors described herein, the first chemical handle comprises an alkyne sugar. In some embodiments of any of the viral vector cores described herein, the viral vector core contains a nucleic acid encoding one or more therapeutic proteins. In some embodiments of any of the viral vector cores described herein, the viral vector core is a lentiviral vector core.

Also provided herein are compositions comprising any of the viral vector cores described herein. Also provided herein are compositions comprising viral-like particle cores, a non-viral enveloped vector cores, or extracellular vesicle cores.

Also provided herein are viral vectors comprising one or more exogenous targeting protein(s), one or more exogenous immune modifying protein(s), and/or one or more polymer(s) covalently attached to its surface through a conjugation signature. In some embodiments of any of the viral vectors described herein, the conjugation signature comprises a triazole bond, a thioether bond, a disulfide bond, or a [4+2] cycloadduct. In some embodiments of any of the viral vectors described herein, the one or more exogenous targeting protein(s) or the one or more exogenous immune modifying proteins have a molecular weight of greater than 2 kDa. In some embodiments of any of the viral vectors described herein, the one or more exogenous targeting proteins comprise an scFv, a nanobody, a darpin, a VHH, an aptamer, or a lectin-binding domain. In some embodiments of any of the viral vectors described herein, the polymer is a polyethylene glycol (PEG). In some embodiments of any of the viral vectors described herein, the PEG has an average molecular weight of about 100 g/mol to about 20000 g/mol. In some embodiments of any of the viral vectors described herein, the viral vector contains a nucleic acid encoding one or more therapeutic protein(s). In some embodiments of any of the viral vectors described herein, the viral vector is a lentiviral vector. In some embodiments of any of the viral vectors described herein, the viral vector has a pseudotype selected from the group of: VSV-G, blinded VSV-G, Sindbis glycoprotein, blinded Sindbis glycoprotein, murine leukemia virus glycoprotein, blinded murine leukemia virus glycoprotein, Moloney murine leukemia virus glycoprotein, blinded Moloney murine leukemia virus glycoprotein, cocal virus glycoprotein, blinded cocal virus glycoprotein, foamy virus glycoprotein, blinded foamy virus glycoprotein. In some embodiments of any of the viral vectors described herein, the viral vector does not have a pseudotype of VSV-G.

Also provided herein are compositions comprising a population of any of the viral vectors described herein. In some embodiments of any of the compositions described herein, the population of the viral vectors has a functional titer that corresponds to greater than about 1E7 TU/mL prior to concentration and conjugation of the viral vector. In some embodiments of any of the compositions described herein, the ratio of the functional titre to the physical titre is about 1:1000 to about 1:10. In some embodiments of any of the compositions described herein, the composition is a pharmaceutical composition.

Also provided herein are methods of making a viral vector that include: (a) contacting a viral vector core comprising on its surface a first chemical handle with an exogenous targeting protein, an exogenous immune modifying protein, or a polymer comprising a second chemical handle, and (b) covalently conjugating the first chemical handle with the second chemical handle, to produce the viral vector. In some embodiments of any of the methods described herein, the method further includes: producing the viral vector core comprising on its surface the first chemical handle. In some embodiments of any of the methods described herein, the step of producing the viral vector core comprises a step of purifying or isolating the viral vector core by clarification and/or chromatography. In some embodiments of any of the methods described herein, the step of producing the viral vector core comprises culturing a producer cell in a liquid culture medium comprising a small molecule capable of metabolically inserting the first chemical handle into glycoproteins on the membrane of the producer cell. In some embodiments of any of the methods described herein, the first chemical handle and the second chemical handle are azide and alkyne. In some embodiments of any of the methods described herein, the first chemical handle and the second chemical handle do not comprise BCN. In some embodiments of any of the methods described herein, step (b) is performed using a click chemistry reaction. In some embodiments of any of the methods described herein, the click chemistry reaction is performed at a temperature of about 30° C. to about 40° C., and a pH of about 6.5 to about 7.5.

Also provided herein are kits including any of the compositions described herein.

Also provided herein are methods of treating a subject in need thereof that include administering to the subject a therapeutically effective amount of any of the compositions described herein.

By the term “viral vector” is meant a modified virus that does not cause the disease associated with the original virus. This viral vector can transduce cells and deliver a transgene (e.g., one or more transgenes). The one or more transgenes can, e.g., encode a therapeutic protein. This viral vector can also deliver a protein. Non-limiting examples of a protein delivered by the viral vector include gene-editing proteins (e.g., Cas9, Cas12a, Cas 12b, Adenine and cytosine base editors, prime editors, and ribonucleoproteins thereof).

By the term “exogenous functional protein” is meant a peptide sequence which can change the function of a viral vector. Non-limiting examples of changed function of a viral vector include targeting, altered interaction with the immune system, immune evasion, and altered biodistribution. In some embodiments, this peptide sequence is not normally present on the surface of the corresponding naturally-occurring viral vector. In some embodiments, this peptide sequence is not normally present in a corresponding naturally-occurring viral vector in the same quantity or form as as when it is introduced to a viral vector as an exogenous functional protein.

By the term “exogenous targeting protein” is meant a peptide sequence which can direct a viral vector to a specific receptor or feature on the surface of a target cell through interaction between the exogenous targeting protein and the receptor or feature on the surface of the target cell. In some embodiments, this peptide sequence is not normally present in a corresponding naturally-occurring viral vector. In some embodiments, this peptide sequence is not normally present on the surface of the viral vector in the same quantity or form as as when it is introduced to a viral vector as an exogenous targeting protein. An exogenous targeting protein is a type of exogenous functional protein.

By the term “exogenous immune modifying protein” is meant a peptide sequence which can alter the way the viral vector would interact with the immune system relative to how it would interact with the immune system in the absence of this protein. In some embodiments, this peptide sequence is not normally present in a corresponding naturally-occurring viral vector. In some embodiments, this peptide sequence is not normally present on the surface of the viral vector in the same quantity or form as as when it is introduced to a viral vector as an exogenous targeting protein. An exogenous immune modifying protein is a type of exogenous functional protein.

By the term “glycoprotein” is meant a molecule expressed on the surface of the viral vector that has one or more carbohydrate group(s) attached to a peptide chain.

By the term “pseudotype” is meant a viral vector with a foreign viral envelope protein not normally associated with the wild type virus.

By the term “producer cells” is meant cells that produce viral vectors or viral vector cores through introduction of plasmids or other nucleic acids into the cells which cause them to produce viral vectors or components of viral vectors. Nucleic acids can be introduced transiently or permanently, the latter being occasionally referred to as a stable cell line in the industry.

By the term “chemical handle” is meant any chemical moiety that can be used for chemical conjugation to a second chemical moiety. Non-limiting examples of chemical handles include click chemical handles (or click handles). Non-limiting examples of click chemical handles are described herein.

By the term “chemical conjugation” is meant the connection of two molecules to each other by covalent or non-covalent chemistries.

By the term “conjugation signature” is meant a bond that remains after covalent conjugation between a first chemical handle and a second chemical handle. A non-limiting example of a conjugation signature includes the bond that remains after covalent conjugation of a first click chemical handle to a second click chemical handle, in which such a conjugation signature can be referred to as a click signature.

By the term “viral vector core” is meant a fragment of a viral vector that contains the genetic material or protein delivery material inside of the viral capsid, without a complete functional envelope including all of the required or desired surface proteins. For example, a viral vector core can lack one or more exogenous targeting protein(s) and/or exogenous immune modifying protein(s).

By the term “functional titre” is meant the concentration of viral vector that has the ability to successfully deliver a transgene to a target cell type. In some embodiments, the viral vector that has the ability to successfully deliver a transgene to a target cell type can have on its surface one or more (e.g., two or more) exogenous targeting protein(s) that allow for the successfully delivery of a transgene to a target cell type. Delivery of a transgene is quantified by titration in either integrating units (IU) or transducing units (TU) based on integrated number of copies and percentage of target cells expressing the transgene at a specific titration, respectively.

By the term “functional viral vector” is meant a viral vector that has the ability to transduce the desired target cells and has all of the desired exogenous functional protein(s) correctly expressed in the desired amount(s).

By the term “non-functional viral vector” is meant a viral vector that does not have the ability to transduce the desired target cells and/or does not have all of the desired exogenous functional protein(s) correctly expressed in the desired amount(s).

By the term “physical titre” is meant the total concentration of viral vector, including both functional and nonfunctional viral vector. Total concentration of viral vector is quantified by genome quantitation or by concentration of viral structural proteins.

By the term “blinded protein” is meant a glycoprotein or cell surface protein which normally binds to a specific target on a cell, but following amino acid modification (e.g., amino acid substitution, deletion, and/or insertion) of the glycoprotein or cell surface protein, no longer binds to that target or has a significantly reduced affinity for binding to the target.

Other definitions appear in context throughout this disclosure. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 is an overview schematic of the LVV production methods described herein, in which plasmids delivered to producer cells enable production of a viral vector core. These viral vector cores are then chemically conjugated with one or more exogenous functional proteins leading to production of a full, functional viral vector.

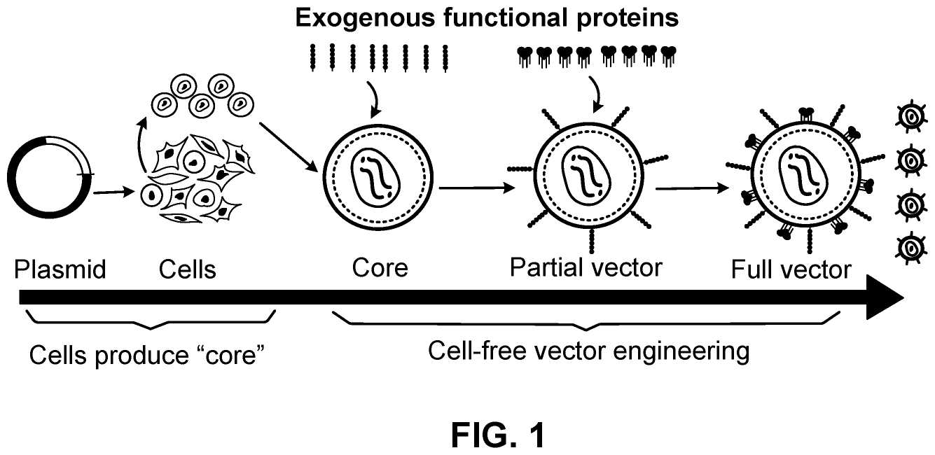

FIG. 2 is a detailed schematic of exemplary LVV production methods described herein in which plasmids are delivered to producer cells to enable production of viral vector cores. These viral vector cores are then chemically conjugated with one or more exogenous functional protein(s) leading to production of a full, functional virus vector. This schematic depicts exemplary plasmid transfection, nuclear localization, transcription, transport to cell membrane, assembly, and budding of the viral vector core, followed by chemical conjugation of one or more exogenous functional protein(s) onto the surface of the viral vector core.

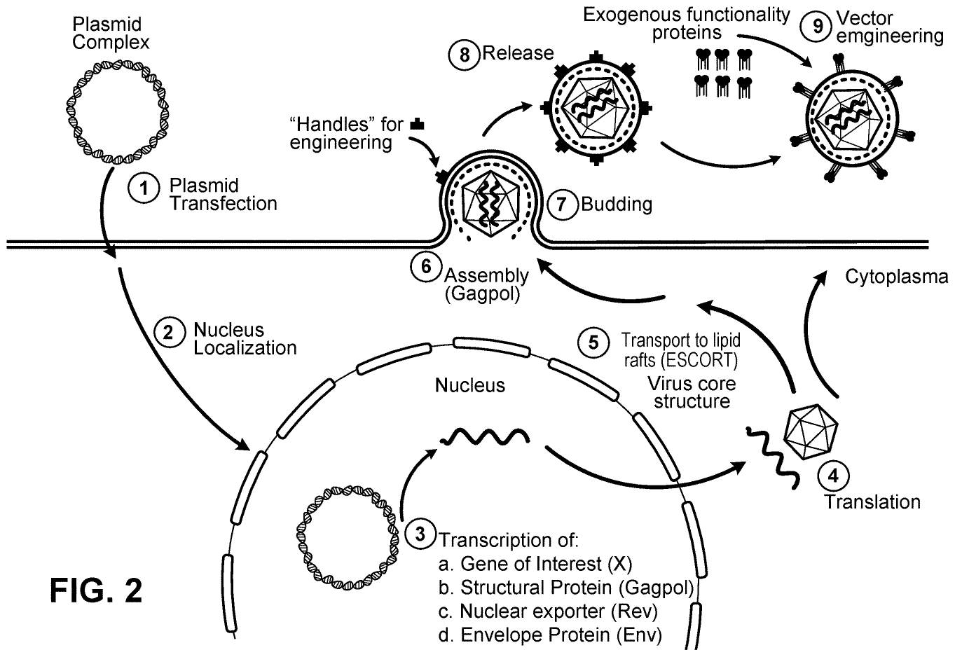

FIG. 3 is a diagram of an exemplary process to produce a therapeutic next generation LVV. An exemplary seed train is used to grow a producer cell culture, which is then transfected with plasmids to generate viral vector cores with the therapeutic transgene. The viral vector cores are then purified and chemically conjugated with the exogenous functional proteins in a cell-free process (in this figure, a CD3-targeting scFv, and two exogenous immune modifying proteins), and formulated for therapeutic administration.

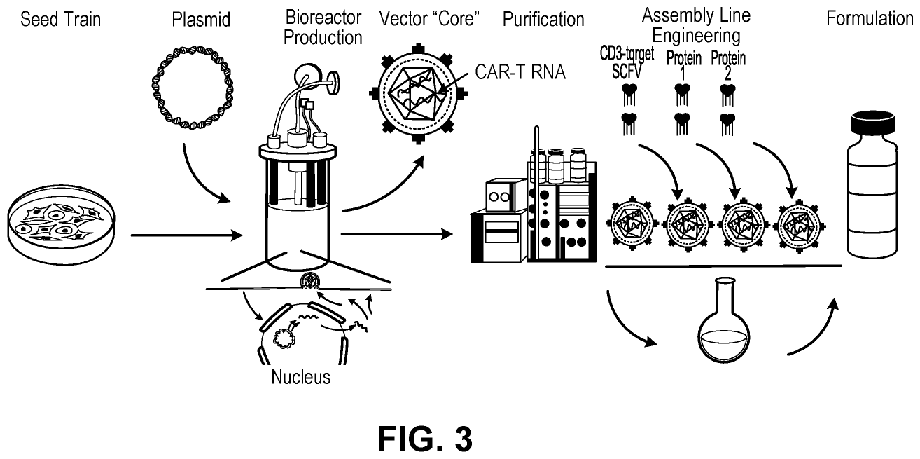

### DETAILED DESCRIPTION

Provided herein are methods for production of a viral vector having exogenous functional protein(s) on its surface. The methods described herein include the steps of contacting a viral vector core comprising on its surface a first chemical handle with an exogenous functional protein(s) comprising a second chemical handle, and covalently conjugating the first chemical handle with the second chemical handle, to produce a viral vector having the exogenous functional protein(s) on its surface. Click chemistries using chemical handles are reviewed in e.g., Devaraj et al., Chemical Reviews special issue on Click Chemistry 121(12): 6697-7248, 2021 and in e.g., Chandrasekaran et al., Click Reactions in Organic Synthesis, Wiley 2016 and in Rutjes et al., Science of Synthesis: Click Chemistry, Thieme, 2021.

In some embodiments, the exogenous functional protein(s) comprise exogenous targeting protein(s). In some embodiments, the exogenous functional protein(s) comprise exogenous immune evasion protein(s). In some embodiments, the exogenous functional protein(s) comprise combinations of exogenous immune evasion protein(s), exogenous targeting protein(s), and exogenous polyethylene glycol(s). In some embodiments, these methods can further include a step of producing the viral vector core. For example, producing the viral vector core can include a step of culturing producer cells (such as HEK293 cells, HEK293T cells, and other cell types) in a medium that supports growth and viral vector production in these cells. Next, plasmid complexes encoding a subset of the attributes of the desired viral vector composition are introduced into the culture media to transfect the producer cells, and the transfected producer cells then produce the viral vector core. In some embodiments, the producer cells are engineered to stably express nucleic acidthat encodes the subset of attributes of the desired viral vector composition that form the viral vector core. In some embodiments, the subset of attributes of the desired viral vector composition that form the viral vector core comprise the viral structural proteins encoded by the gagpol sequence, the regulatory protein encoded by the rev sequence, the transgene(s) of interest encoded by a specific nucleic acid sequence for that protein or proteins (e.g., therapeutic protein or therapeutic proteins). In some embodiments, the viral vector core further comprises the glycoprotein that mediates internalization by fusion to a target cell membrane or by fusion to an endosome membrane. In some embodiments, a first chemical handle is introduced into the culture media (such as an azido sugar), which the producer cell integrates into cellular components. In some embodiments, these components comprise glycoproteins that localize to the surface of the producer cell. When a viral vector core buds off the producer cell, the surface of the viral vector core comprises on its surface the glycoproteins into which a first chemical handle has been integrated (e.g., the glycoproteins comprise the first chemical handle or multiple copies of the first chemical handle, or the glycoproteins have a covalently attached first chemical handle or have multiple copies of a covalently attached first chemical handle). Some embodiments of these methods further include a step of producing the exogenous functional protein comprising the second chemical handle. For example, the second chemical handle can comprise an alkyne moiety (DBCO, BCN, etc). Click chemistry reactions and suitable chemical groups for click chemistry reactions that can comprise the first and second handle are well known to those of skill in the art, and include, but are not limited to terminal alkynes or alkynyl groups, azides or azido groups, strained alkynes or alkynyl groups, tetrazine groups including substituted forms thereof, dienes, dieneophiles, alkoxyamines or alkoxyamino groups, carbonyls, phosphines. hydrazides, hydrazines, hydrazones, thiols, and alkene moieties. In non-limiting embodiments, the click reaction does not require a catalyst. In non-limiting embodiments, the click reaction does not require copper ions, e.g., proceeds at substantially the same rate in the absence of copper ions as in the presence of copper ions.

When a chemical handle can participate in a click chemistry reaction, the chemical can be referred herein as a click handle. For example, a strained cycloalkene, e.g., a cyclooctene, is a click handle, since it can partake in a strain-promoted cycloaddition that is a non-limiting example of a click chemistry reaction. In general, click chemistry reactions require at least two molecules (a click-pair) comprising click handles that can react with each other. Within a click-pair, a first click handle can provide a nucleophilic (Nu) group and a second click handle can provide an electrophilic (E) group that can react with the Nu group of the first click handle (or vice versa). Such click-pairs that are reactive with each other are sometimes referred to herein as partner click handles. For example, a tetrazine is a partner click handle to a cyclooctene or any other alkene. In this way, click-pairs can be selected and provided on separate entities (entity A and entity B), such that providing both entities A and B will result in the click handles of the click-pair to react.

Exemplary click handles suitable for use according to some aspects of this disclosure are described herein. In some embodiments, the click handles of a click-pair can include (i) a terminal alkyne and an azide; (ii) a strained alkyne and an azide; (iii) a diene and a dieneophile; (iv) an alkoxyamine and a carbonyl; (v) a phosphine and an azide; (vi) a hydrazide and a carbonyl; (vii) a thiol and an alkene; (vii) a cyclooctyne (e.g., a strained cyclooctyne (SCO), such as difluorooctyne (DIFO), dibenzylcyclooctyne (DIBO), biarylazacyclooctynone (BARAC), or bicyclo[6.1.0]nonyne (BCN)) and an azide; or (viii) a cyclooctene (e.g., trans-cyclooctene (TCO) and a tetrazine. Additional click handles are described in Becer et al., Angew. Chem. Int. ed. 2009:48:4900-4908, and Int. Pub. No. WO 2013/003555 and references therein, which references are incorporated herein by reference for click handles and methodology.

When both members of a click-pair are present, the click handles will react, and a click conjugation signature (or click signature) can form. In some embodiments, the click signature has the structure of a click signature described herein.

A click handle can further comprise a linker or a linker region. Exemplary linker regions are described herein. When linker regions are present, a click signature can be present within the residual linker formed as a result of the click reaction.

In some embodiments, the click signature comprises a cyclic moiety, e.g., a heterocycle such as triazole e.g., a disubstituted triazole, or a cycloadduct. In some embodiments, the click signature comprises a cycloalkene such as cyclohexene, an alkyl sulfide, a dihydropyrazine such as a 1,2-dihydropyrazine, a diazole, or a sulfur-containing ring such as a thiopyran.

In some embodiments, the click-pair includes a first click handle and a second click handle that reacts with that first click handle. Exemplary click handle include, e.g., a click-chemistry group, e.g., one of a click-pair selected from the group consisting of a Huisgen 1,3-dipolar cycloaddition reaction between an alkynyl group and an azido group to form a triazole-containing linker; a copper(I)-catalyzed azide-alkyne cycloaddition reaction between an alkynyl group and an azido group to form a triazole-containing linker; a strain-promoted azide-alkyne cycloaddition (SPAAC) reaction between a strained alkynyl group (e.g., present in a strained cyclooctynyl group, such as DIFO, DIBO, or others described herein) and an azido group to form a triazole-containing linker; a strain-promoted alkyne-nitrone cycloaddition (SPANC) reaction between a strained alkynyl group (e.g., present in a strained cyclooctynyl group, such as DIFO, DIBO, or others described herein) and a nitrone group to form an isoxazoline-containing linker; a Diels-Alder reaction (including an inverse electron demand Diels-Alder reaction) between a diene having a 4π electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and a dienophile or heterodienophile having a 2π electron system (e.g., an optionally substituted alkenyl group or an optionally substituted alkynyl group); a ring opening reaction with a nucleophile and a strained heterocyclyl electrophile; a splint ligation reaction with a phosphorothioate group and an iodo group; a Staudinger reaction with a phosphine and an azide; and a reductive amination reaction with an aldehyde group and an amino group.

In some embodiments, the click signature was formed by or is capable of being formed by cycloaddition (e.g., a 1,3-dipolar cycloaddition or hetero-Diels-Alder cycloaddition), nucleophilic ring-opening (e.g., openings of strained heterocyclic electrophiles such as aziridines, epoxides, cyclic sulfates, aziridinium ions, and episulfonium ions), carbonyl chemistry of non-aldol type (e.g., formation of ureas, thioureas, hydrazones, oxime ethers, amides, or aromatic heterocycles), or an addition to a carbon-carbon multiple bond (e.g., epoxidation, aziridination, dihydroxylation, sulfenyl halide addition, nitosyl halide addition, or Michael addition). Examples of these types of click reaction are described in greater detail in Hein et al., Pharm. Res. 2008 October; 25(10): 2216-2230, which is herein incorporated by reference in its entirety. In embodiments, the click reaction is a metal-free [3+2] cycloaddition reaction, Diels-Alder reaction, or thiol-alkene radical reaction. Examples of these types of click reaction are described in greater detail in Becer et al., Angew. Chem. Int. Ed. 2009, 48, 4900-4908, which is herein incorporated by reference in its entirety.

In an embodiment, the click signature is an alkyne/azide click signature (e.g., wherein the alkyne is a cyclooctyne, activated alkyne, or electron-deficient alkyne), e.g., the click signature comprises a triazole, e.g., a 1,2,3-triazole and/or a disubstituted triazole. In an embodiment, the click signature is a diene/dienophile click signature (e.g., wherein the dienophile comprises an alkene moiety), e.g., the click signature comprises a cycloalkene, e.g., a disubstituted alkene. In embodiments, the click signature is a tetrazine/alkene click signature, e.g., the click signature comprises a dihydropyrazine, e.g., a 1,2-dihydropyrazine. In embodiments, the click signature is a tetrazole/alkene click signature, e.g., the click signature comprises a diazole. In embodiments, the click signature is a dithioester/diene click signature, e.g., the click signature comprises a sulfur-containing ring, e.g., a tetrahydrothiophene, e.g., a disubstituted tetrahydrothiophene. In embodiments, the click signature is a dithioester/diene signature, e.g., the click signature comprises a sulfur-containing ring, e.g., a thiopyran. In embodiments, the click signature is a thiol/alkene click signature, e.g., the click signature comprises an alkyl sulfide.

One example of a cycloaddition reaction is the Huisgen 1,3-dipolar cycloaddition of a dipolarophile with a 1,3 dipolar component that produce five membered (hetero)cycles. Examples of dipolarophiles are alkenes, alkynes, and molecules that possess related heteroatom functional groups, such as carbonyls and nitriles. Specifically, another example is the 2+3 cycloaddition of alkyl azides and acetylenes. Other cycloaddition reactions include Diels-Alder reactions of a conjugated diene and a dienophile (such as an alkyne or alkene). Examples of cycloaddition reactions are described, e.g., in U.S. Pat. No. 9,517,291, which is herein incorporated by reference in its entirety.

Other examples of click reactions include a hydrosilation reaction of H-Si and simple non-activated vinyl compounds, urethane formation from alcohols and isocyanates, Menshutkin reactions of tertiary amines with alkyl iodides or alkyl trifluoromethanesulfonates, Michael additions, e.g., the very efficient maleimide-thiol reaction, atom transfer radical addition reactions between —SO₂Cl and an olefin (e.g., including a carbon-carbon double bond), metathesis, Staudinger reaction of phosphines with alkyl azides, oxidative coupling of thiols, nucleophilic substitution, especially of small strained rings like epoxy and aziridine compounds, carbonyl chemistry like formation of ureas, and addition reactions to carbon-carbon double bonds like dihydroxylation. Therefore, attached functionality may be chosen from acetylene bond, an azido-group, a nitrile group, acetylenic, amino group, phosphino group. The click chemistry reaction may result in the addition of a functional group selected from amino, primary amino, hydroxyl, sulfonate, benzotriazole, bromide, chloride, chloroformate, trimethylsilane, phosphonium bromide, and the like.

Further click reactions, click handles, and process conditions are described elsewhere, e.g., Kolb et al., Angew. Chem. Int. Ed. 2001; 40:2004-2021; Evans, et al., Austral. J. Chem. 2007; 60:384-395; Carlmark et al., Chem. Soc. Rev. 2009; 38:352-362; U.S. Pat. No. 8,912,323; and Lahann, Click Chemistry for Biotechnology and Materials Science, 2009, John Wiley & Sons Ltd, ISBN 978-0-470-69970-6, each of which is incorporated herein by reference in its entirety.

In some embodiments, the first and second chemical handle can comprise either an azide or an alkyne. In some embodiments, the first and second chemical handle can comprise either a maleimide or a thiol. In some embodiments, the thiol is the side chain of the amino acid cysteine. In some embodiments, the first and second chemical handle can comprise either a diene or a dienophile. In some embodiments, the diene is a tetrazine. In some embodiments, the dienophile is an olefin. In some embodiments, the dienophile is trans-cyclooctene. In some embodiments, the first and second chemical handle can comprise one of Protein A, Protein G, Protein AG, and an antibody. In some embodiments, the first and second handle can comprise a SNAP tag and a benzylguanine derivative. In some embodiments, the first and second handle can comprise a CLIP tag and a benzylcytosine derivative.

In some embodiments the first and second chemical handle can comprise a biotin, a monomeric streptavidin, a monomeric streptavidin 2, an intein, a SunTag, an Isopeptag, a Spy Tag, a SpyCatcher, a SnoopTag, a SnoopTagJr, a SnoopCatcher, a DogTag, a DogCatcher, a Glutathione-S-transferase, a CLIP, an HA, a FLAG, and a HiBiT.

In some embodiments, the first and second chemical handle do not comprise BCN, Protein A, Protein G, Protein AG, Spytag, or Spycatcher.

In some embodiments, the first and second chemical handle do not comprise SNAP tag, benzylguanine, alkyne or alkylnyl, azide or azido, and/or phosphine or phosphinyl.

In some embodiments, the first and second chemical handle do not comprise cyclooctene or cyclooctenyl, cyclooctyne or cyclooctynyl, tetrazine or tetrazinyl (e.g., 1,2,4,5-tetrazine or 1,2,4,5-tetraziny1), tetrazole or tetrazolyl (e.g., 2-tetrazole or 2-tetrazolyl), phosphine or phosphinyl (e.g., triphenylphosphine or triphenylphosphinyl), aldehyde, ketone, alkanoyl (e.g., —C(O)R, in which R is an organic substituent, such as alkyl or an aliphatic group), formyl (e.g., —C(O)H), hydrazine or hydrazino (e.g., —NHNH₂), and/or hydroxylamine or hydroxylamino (e.g., —ONH₂).

In some embodiments, the first chemical handle is embedded in the lipid bilayer membrane of the producer cell or in the lipid bylayer membrane of the vector.

In some embodiments, the first chemical handle is not embedded in the lipid bilayer membrane of the producer cell or in the lipid bylayer membrane of the vector.

In some embodiments, the first chemical handle is embedded in glycoproteins on the surface of the producer cell and on the surface of the membrane of the vector.

In some embodiments, the first chemical handle is not embedded in glycoproteins on the surface of the producer cell and on the surface of the membrane of the vector.

The produced exogenous targeting protein comprising the second chemical handle can optionally be purified and prepared in specific stoichiometries for conjugation to the first chemical handle(s) of the viral vector core. The conjugation reaction can be performed, e.g., under physiological conditions (e.g., at a temperature of 30-40° C. (e.g., 37° C.) or at a pH of about 6.5 to 7.5 (e.g., pH 7.3)). Unlike other methods of genetic modification to proteins on the viral envelope, this method introduces as few as 3 atoms at each conjugation site, and minimal negative physiological impact to the viral vector is anticipated. Additional non-limiting aspects of these methods are described herein.

A non-limiting example of preparation of an exogenous targeting molecule with a chemical handle involves first digesting an antibody with an IdeS, IdeZ, or papain enzyme. Next, a molecule comprising a maleimide and an additional chemical handle is reacted with the reduced antibody fragment.

Also provided herein are viral vectors produced by these methods, populations of viral vectors produced by these methods, compositions (e.g., pharmaceutical compositions) comprising any of the viral vectors or populations of viral vectors described herein, and kits comprising any of the compositions described herein.

Also provided herein are methods of administering any of the viral vectors described herein or any of the compositions (e.g., pharmaceutical compositions described herein) to a subject (e.g., a human) in need thereof.

### Methods for Production of Viral Vector Cores

Production of viral vector cores begins with culturing a producer cell in a liquid culture medium comprising a small molecule capable of metabolically inserting the first chemical handle into proteins (e.g., glycoproteins) in the plasma membrane of the producer cell. Several producer cell types are known in the art for production of viral vectors. In some embodiments, the producer cells are HEK293 cells. In some embodiments, the producer cells are HEK293T cells. Several liquid culture media are known in the art for production of viral vectors. In some embodiments, the liquid culture medium is LVMAX medium (Thermo Fisher). Several small molecules capable of metabolically inserting a first chemical handle into a protein (e.g., glycoprotein) or into the lipid bylayer in the plasma membrane of a producer cell are known in the art. In some emboidements, this small molecule is an azido sugar peracetylated N-α-azidoacetylmannosamine (Ac₄ManNAz).

This method can be used to produce viral vector cores derived from many viral vector types. The examples herein describe viral vector cores and viral vectors wherein the viral vector is a lentiviral vector. In some embodiments, the viral vector can be a retroviral vector, alphavirus vector, filovirus vectors, a rhabdoviral vector, a Moloney murine leukemia vector, or resporiatory syncytial viruses. In some embodiments, the viral vector is an enveloped viral vector. In some embodiments, the viral vector is an extracellular vesicle.

The liquid culture medium can optionally be supplemented with glucose or other common additives known in the art to support general cell culture.

Viral vector cores can be produced from cultured producer cells by introducing a transfection complex to the cell culture in a method known in the art as transient transfection. In this method, a plasmid complex is formed by combining plasmids in specific ratios with a complexation agent in a separate liquid medium. In some embodiments, the plasmids included in the complex comprise a plasmid encoding the viral structural proteins gagpol, a plasmid encoding the regulatory protein rev, and a plasmid encoding the transgene(s) of interest (known in the art as a third generation lentiviral vector system). Third generation lentiviral vector systems are described, e.g., in Dull et al., J. Virol. 72(11): 8463-71, 1998 and e.g., U.S. Pat. No. 8,329,462 B2. In some embodiments, the plasmid encoding the transgene(s) of interest contains a nucleic acid encoding one or more therapeutic proteins.

In some embodiments, the complex further comprises a plasmid encoding a glycoprotein. The glycoprotein encoded by the glycoprotein plasmid in this example can fuse a viral vector to a cell membrane or an endosome membrane, but lacks tropism to human cells, either due to a natural lack of tropism to human cells or due to mutation removing the tropism of a glycoprotein that would otherwise have tropism to human cells. In some embodiments, the glycoprotein is a vesicular stomatitis virus glycoprotein (VSV-G), a Sindbis virus glycoprotein, a murine leukemia virus glycoprotein, a Moloney murine leukemia virus glycoprotein, a cocal virus glycoprotein, a foamy virus glycoprotein, a retroviral family glycoprotein, a GP64 glycoprotein, a rhabdovirus family glycoprotein, a filovirus family glycoprotein, or a paramyxovirus family glycoprotein. In some embodiments, the glycoprotein is blinded to its native receptor. For example, blinded VSV-G is described, e.g., in US20200216502 A1, blinded Sindbis virus is described, e.g., in U.S. Pat. No. 7,429,481 B2, and blinded paramyxovivurs is described, e.g., in US20220064674 A1

In some embodiments, polyethylenimine (PEI) is used as a complexation reagent. Additional transfection agents and transfection methods are known in the art. When the producer cells reach the target density of 3E 6 (range of 5 E5 to 5E7) at the production volume and vessel, the plasmid complex is added to the cell culture. In some embodiments, 24 hours (range 12 h-48 h) after addition, the liquid culture medium is supplemented with a transfection enhancing molecule, such as sodium butyrate. Additional transfection enhancing molecules are known in the art.

In some embodiments, viral vector cores can be produced from producer cells cultured as described above by a method in which one or more of the genes encoded by the plasmids described above are stably introduced into the producer cells. In some embodiments, these engineered producer cells can produce viral vector cores without introduction of plasmid complexes. In some embodiments, these engineered producer cells can produce viral vector cores following introduction of plasmid complexes that comprise a subset of the plasmids described above for the transient transfection method.

These methods result in production of a viral vector core comprising on its surface the first chemical handle (e.g., one or more copies of the first chemical handle).

48 hours after complex addition (range 24 h-96 h), a step of purifying or isolating the viral vector core by centrifugation, clarification, or chromatography can be performed. The clarification step removes cells and cellular debris. In some embodiments, a chromatography step is performed which removes protein and DNA impurities. In some embodiments, a concentration step is performed by centrifugation or filtration. In some embodiments, buffer exchange is performed. Several concentration, filtration, and buffer exchange methods are known in the art. At this stage in the process, a viral core has been produced, purified, and concentrated.

### A Viral Vector Core Having Covalently on to Its Surface a First Chemical Handle

A viral vector core is characterized for physical titre and presence of the first chemical handle. The physical titre assays used are ELISA for the p24 lentiviral structural protein and PCR for vector genome quantitation. These assays and methods for calculating titer from the assays are known in the art.

The methods to produce a viral vector core can be characterized by applying these physical titre assays at or during several steps in the method. Prior to the step comprising purifying or isolating the viral vector core by clarification and/or chromatography, the liquid culture medium is sampled and assayed for physical titre.

### Exogenous Functional Proteins

Exogenous targeting protein(s), exogenous immune modifying protein(s), and polymers are prepared to be covalently attached to the surface of the viral vector core by chemical conjugation. These exogenous functional proteins and polymers endow the viral vector with unique functional properties.

Exogenous functional protein(s) (e.g., exogenous targeting protein(s) and/or exogenous immune modifying protein(s)) and/or polymer(s) (e.g., polyethylene glycol(s)) are synthesized with a second chemical handle which reacts with a first chemical handle present on the viral vector core. Several chemical handles and methods for synthesizing proteins comprising them are known in the art. In some embodiments, a second chemical handle is dibenzocyclooctyne (DBCO) or bicyclo[6.1.0]nonyne (BCN). In some embodiments, a second chemical handle is not BCN. A non-limiting example of preparation of an exogenous targeting molecule with a chemical handle involves first digesting an antibody with an IdeS, IdeZ, or papain enzyme. Next, a molecule comprising a maleimide and an additional chemical handle is reacted with the reduced antibody fragment.

In some embodiments, the exogenous targeting proteins comprise a second chemical handle synthesized onto an Antibody, antibody fragment, single chain variable fragments (scFv), a nanobody, a darpin, a VHH, an aptamer, or a lectin binding domain that bind to surface molecules unique to the target cells (e.g., T cells or B cells). T cell targeting molecules are described, e.g., in U.S. Pat. No. 8,784,821 B1. Non limiting examples of antibody fragments include F(ab′)2, Fab, Fab′, Bispecific Fab2, bispecific antibodies, trispecific Fab3, minibodies, scFv-Fc, and diabody.

A polymer can optionally be conjugated to the viral vector core. In some embodiments, polyethylene glycol (PEG)-DBCO is conjugated to a viral vector core. In some embodiments, the PEG has an average molecular weight of about 100 g/mol to about 20000 g/mol. Methods for chemical conjugation of PEG to viral vectors are known in the art. See, e.g., U.S. Pat. No. 6,399,385 B1.

These exogenous functional proteins and polymer(s), having a second chemical handle, can be mixed at defined stoichiometric ratios in solution.

### Methods for Conjugating a Viral Vector Core With Function-Defining Proteins and Molecules

In this method of making a viral vector, a viral vector core comprising on its surface a first chemical handle (e.g., one or more copies of a first chemical handle) is contacted with an exogenous targeting protein(s), an exogenous immune modifying protein(s), and/or a polymer(s) comprising a second chemical handle. Contacting the first chemical handle with the second chemical handle covalently conjugates the exogenous functional protein(s) and/or polymer(s) to the viral vector core to produce the viral vector.

In some embodiments, the exogenous functional protein(s) or polymers comprising a second chemical handle are introduced into the liquid culture medium into which the viral vector core has been produced by the producer cells. In some embodiments, the liquid culture medium has been subjected to a step comprising a step of purifying or isolating the viral vector core by clarification and/or chromatography prior to introduction of the exogenous functional protein(s) and/or polymer(s) comprising a second chemical handle. In some embodiments, the liquid culture medium has been subjected to a concentration and/or buffer exchange step prior to introduction of the exogenous functional protein(s) and/or polymer(s) comprising a second chemical handle.

The exogenous functional protein(s) and/or polymer(s) are introduced at physiological conditions (pH about 6.5 to about 7.5; temperature about 30° C. to about 40° C.) for 1 hour (about 30 min to about 24 hours). In some embodiments, the liquid culture medium is mixed. Following this step, a viral vector is produced with one or more exogenous targeting protein(s), one or more exogenous immune modifying protein(s), and/or one or more polymer(s) covalently attached to its surface or components on its surface through a conjugation signature. In some embodiments, the conjugation signature comprises a triazole bond. In some embodiments, the conjugation signature comprises a 1-2-3 triazole bond. In some embodiments, the conjugation signature is only a 1-2-3 triazole bond.

In some embodiments, the conjugation signature comprises a thioether bond. In some embodiments, the conjugation signature comprises a disulfide bond.

In some embodiments, the conjugation signature comprises a [4+2] cycloadduct.

In some embodiments, a next step comprising purification of the viral vectors by chromatography is performed to remove unreacted substrates. Additional processing steps may be performed depending on the required application.

### Viral Vectors With Exogenous Functional Properties Produced With Chemical Conjugation

Viral vectors with one or more exogenous targeting protein(s), one or more exogenous immune modifying protein(s), and/or one or more polymer(s) covalently attached to its surface through a conjugation signature have several unique functional and physical properties.

Most importantly, the production of a viral vector with a single exogenous targeting protein while maintaining a high functional titre has been a major challenge of the gene therapy industry. Viral vectors with other exogenous functional proteins (such as exogenous immune modifying proteins) have shown beneficial properties; however, the current industry standard production methods have not permitted for production of a viral vector with multiple exogenous functional proteins. A viral vector with multiple exogenous functional proteins at titres relevant for therapeutic use has never previously been described. This has limited the gene therapy industry from making viral vectors with more advanced functions and has limited the industry's ability to extend this modality into additional therapeutic areas. Notably, the method described herein results in a pharmaceutical compositions of viral vectors with exogenous functional proteins and/or polymers at functional titres significantly greater than viral vectors with exogenous functional proteins produced without using the methods described herein. This results in improved delivery efficiency. This unlocks significant therapeutic areas for these therapies that today's gene therapy industry cannot access.

The physical titre is similar between the methods of producing viral vectors described herein and previous methods of producing viral vectors. Hence, the methods provided herein provide for a population of viral vectors significantly enriched for functional viral vectors. The viral vectors produced by the methods described herein therefore represent both a more potent pharmaceutical composition and a pharmaceutical composition with more advanced function than any that has been described previously.

Functional titre is assessed in vitro using a titre assay that is known in the art. In brief, solutions containing viral vectors are serially diluted and a recipient cell line or primary cell is transduced. Transduction is assessed at each dilution point, and IU and TU/mL are calculated. In some embodiments, the jurkat cell line is used as well as primary T cells for titering. In some embodiments, HEK293 cells engineered to overexpress the target of the exogenous targeting protein is used for titering.

The ratio of functional titre to physical titre is a measure of what fraction of the viral vectors are functional.

The pharmaceutical compositions described herein can be delivered intravenously to mice and nonhuman primates. In these animals, the viral vector transduces the target cells in vivo, and evades recognition by the immune system due to the exogenous immune evasion proteins and exogenous PEG. The exogenous targeting proteins mediate binding to specific cell receptors on the target cells upon interaction with that cell type in the animal. In some embodiments, alternate exogenous targeting molecules mediate binding to other specific cellular receptors upon interaction with alternate cell types. In some embodiments, alternate exogenous functional proteins elicit specific interactions or inhibit specific interactions in vivo. Upon binding of the viral vector to the target cell, the viral vector either fuses to the cell membrane or is internalized into an endosome and fuses with the endosome membrane, as mediated by the glycoprotein. This initiates a well-characterized process of viral transduction, resulting in the insertion of the transgene into the target cell in the animal. In some embodiments, the transgene encodes for green fluorescent protein (GFP).

### Pharmaceutical Compositions and Clinical Applications

The ability to target specific cell types in humans and modify their genes using viral vectors conjugated with exogenous targeting proteins would transform current practices in many medical fields. In some embodiments, viral vectors chemically conjugated with exogenous targeting proteins that target T cells in vivo and transduce them with a therapeutic protein could enable the generation of therapeutic cancer targeting cells directly in the body of human patients. This would transform current medical practice in oncology and many other severe diseases (including autoimmune diseases and others). Today, several cancer-targeting cell therapies are approved which have efficacy in patients with severe and terminal blood cancers. However, this field struggles because these therapies are manufactured in a patient-specific fashion, which has restricted access to these therapies and led to many patient deaths while waiting for manufacturing. Furthermore, these drugs are very expensive to produce, and patient access outside of select geographies is extremely limited.

The viral vectors described herein could create a new and transformative means for treating these same cancer patients and others. In some embodiments, the pharmaceutical compositions with high functional titres described herein could generate cancer-targeting cells and other therapeutic cells directly in the body and bypass the challenging manufacturing and treatment required for today's advanced cell therapies. In some embodiments, the viral vectors described herein could comprise exogenous targeting proteins that target the viral vector to any cell in the body and deliver a nucleic acid encoding one or more therapeutic proteins. In some embodiments, these genes are integrating genes leading to durable expression or nonintegrating genes for transient expression.

Enabled by this method to produce viral vectors with one or more exogenous functional proteins in populations enriched for functional viral vectors, these methods and the viral vectors, viral vector populations, and pharmaceutical compositions could lead to many patient cures for prevalent diseases that the current state of the art viral vector therapies could not treat.

Provided herein are pharmaceutical compositions that include a population of viral vectors produced by any of the methods described herein. In some embodiments, the pharmaceutical compositions comprise a functional titer of at least about 1E6TU/mL, at least about 1E7TU/mL, at least about 1E8 TU/mL, at least about 1E9 TU/mL, or at least about 1E10 TU/mL. In some embodiments, the pharmaceutical compositions comprise a functional titer to physical titer ratio of at least about 1:10, at least about 1:100, or at least about 1:1000.

### Kits for Production of Viral Vectors

The present disclosure contemplates kits for carrying out the methods provided herein. Such kits typically comprise two or more components required for production of viral vectors. Components of the kit include, but are not limited to, one or more of compounds, reagents, containers, equipment, and instructions for using the kit. Accordingly, the methods described herein may be performed by utilizing pre-packaged kits provided herein.

In some embodiments, a kit for production of viral vectors is provided. The kit comprises one or more exogenous functional protein(s) and/or polymer(s) comprising a second chemical handle. In some embodiments, the kit further comprises one or more medium components of each of a liquid culture medium, a growth supplement, a small molecule capable of metabolically inserting a first chemical handle into a producer cell, and a transduction enhancer. In some embodiments, the kit further comprises a producer cell. In some embodiments, the kit further comprises all of the above components.

In some embodiments, instructions for use of the kit to produce a viral vector is provided. The instructions may comprise one or more protocols for: liquid culture media formulations; culture conditions, such as time, temperature, and/or gas incubation concentrations; transfection protocols; harvesting protocols; and protocols for identifying viral vectors. The kit may further include materials useful to conduct the present method including culture plates, welled plates, flasks, chromatography columns, filters, petri dishes and the like.

Also provided herein are kits that include a population of viral vector cores comprising a first chemical handle and one of more exogenous functional proteins that comprise a second chemical handle and/or one or more PEGs that comprise a second chemical handle. In some embodiments, the kits further comprise instructions for performing a chemical conjugation reaction between the first and second chemical handles.

Also provided herein are kits that include a composition (e.g., a pharmaceutical composition) comprising any of the viral vectors described herein (e.g., viral vectors produced by any of the methods described herein).

The invention is further described in the following examples, which do not limit the scope of the invention described in the claims.

### EXAMPLES

### Example 1. Production of a Viral Core With an Azide Handle

In Example 1, a viral vector core with a first chemical handle is produced. In this example, the first chemical handle is an azide handle.

Viral vector core production follows the LVMAX viral production system manufacturer guidelines (see, LVMAX viral production system manual on the ThermoFisher website), summarized below with modifications noted.

HEK 293 cells are seeded into in a 125 mL shake flask in liquid culture medium (LVMAX medium, Thermo Fisher). Ac₄ManNAz is added into the liquid culture medium at a concentration of 50 μM. Cells are counted daily using a ViCell counter. When the cell density reaches 3 million cells/mL, transfection is performed.

A transfection complex is formed by combining the gagpol, rev, GFP, and glycoprotein plasmids together. 1.5 μg of total plasmid DNA/mL is added to OptiMem medium (Thermo Fisher), to a defined ratio of the above plasmids. PEI pro (Polyplus) is added to the OptiMem medium at 1.5 μg/mL. The total volume of the complexation reaction is 10% of the total culture volume. The complexation reaction takes 10 minutes, following which the complexation mixture is added to the liquid culture medium.

24 hours after transfection, sodium butyrate (Sigma) is added to the liquid culture medium at 1 mM. 48 hours after transfection, the viral vectors are harvested by clarification through a 0.45 μm filter (Pall). The viral vectors are purified through an anion exchange chromatography column (Pall). Alternatively, vectors are concentrated via ultracentrifugation and resuspension in buffer.

A sample is taken prior to clarification, after clarification, and after chromatography (or prior to and after ultracentrifugation). The sample is assayed for physical and functional titre. The physical titre is about 1000 ng/ml (approximately 1.25E10 particles/mL) prior to clarification. Less than 25% loss of physical titre is observed after clarification. A further loss of less than 30% is observed after chromatography. Functional titre of all samples is below the limit of detection. This is expected since the viral vector core lacks the ability to transduce cells.

Host cell protein and host cell DNA are assayed from these three samples using commercially available kits (Thermo Fisher). A reduction of host cell protein is observed following clarification and chromatography.

### Example 2. Production of an Exogenous Targeting Protein With an Alkyne Handle

In Example 2, an exogenous targeting protein with a second chemical handle is produced. In this example, the exogenous targeting protein is a F(ab′) that targets a protein on the surface of T cells (e.g., CD3). In this example, the second chemical handle is an alkyne handle.

An The exogenous targeting protein with alkyne handle is prepared by first digesting a CD3 antibody with an IdeS enzyme. Next, a DBCO-PEG-Maleimide molecule is reacted with the thiol group on the F(ab′) to form the exogenous functional protein comprising the second chemical handle. Optionally, reverse phase hPLC or size exclusion chromatography is used to purify.

The resulting protein is assayed by SDS Page to confirm a protein band is observed in the expected size range.

### Example 3. Production of an Exogenous Immune Modifying Protein With an Alkyne Handle

In Example 3, an exogenous immune modifying protein with a second chemical handle is produced. In this example, the exogenous immune modifying protein is the extracellular domain of CD47. In this example, the second chemical handle is an alkyne handle. CD47 is known in the art to be an immune modifying protein for viral vectors. See, e.g., U.S. Pat. No. 9,050,269 B2.

A CD47 protein is produced using protein production methods known in the art. DBCO-PEG5-NHS is reacted with the NH2 groups on the protein to form the exogenous functional protein comprising the second chemical handle. Optionally, reverse phase hPLC or size exclusion chromatography is used to purify.

The resulting protein is assayed by SDS-PAGEto confirm a band is observed in the expected size range.

### Example 4. Production of a Viral Vector With an Exogenous Immune Modifying Protein and/or an Exogenous Targeting Protein

In Example 4, a viral vector is produced which comprises the CD47 exogenous immune modifying protein or the CD3-targeting scFv exogenous targeting molecule or both on the surface and a nucleic acid encoding a GFP protein.

The exogenous functional proteins of Examples 2 and 3 are introduced into the medium comprising the viral vector core of example 1 either separately or together. The reaction occurs in an incubator at 37° C. with physiological pH of 7.3 for 1 hour.

The resulting viral vector is assayed for functional titre as described above on jurkat cells, which express CD3 as well as HEK293 cells engineered to overexpress CD3. Relative to viral vectors with these same exogenous functional proteins produced using traditional viral vector production methods, the functional titre of the resulting viral vector using the methods described herein is expected to be significantly higher.

This study provides for the generation of a T-cell targeting enveloped vector, an immune-modified enveloped vector, and the combination of a T-cell targeting molecule and an immune modifying molecule on a vector while maintaining a functional titre of >1E8 TU/mL. Accounting for the concentration steps in producing this preparation, this corresponds to an unconcentrated titre of >1E6 TU/mL. Previously, functional titres for T-cell targeting lentiviral vectors have been <1E6 TU/mL as described in, e.g., Bender, R. R., et al (2016) Receptor-Targeted Nipah Virus Glycoproteins Improve Cell-Type Selective Gene Delivery and Reveal a Preference for Membrane-Proximal Cell Attachment.” PLOS Pathogens 12(6): e1005641. Other T-cell targeting viral vectors have been described, e.g., in US20220064674 A1. This study provides for a lentiviral vector preparation with greater than 10 fold higher functional titre. Noting that the physical titre is expected to be similar between this method of preparation and those described previously, the population of viral vectors resulting from this method is expected to be enriched by about 10 fold in functional viral vectors. Delivery efficiency is expected to be increased with this vector relative to vectors produced without the methods described herein.

In some embodiments, this viral vector can be further conjugated to PEG as described in, e.g., U.S. Pat. No. 6,399,385 B1. In some embodiments, the viral vector generated in this and the previous examples excludes PEG.

### Example 5. Characterization of Exogenous Functional Proteins Present on Viral Vectors

In Example 5, the viral vectors produced in Example 4 are characterized to confirm the exogenous functional proteins are present on the viral vector.

The viral vectors of example 4 are stained with antibodies against the CD3 targeting molecule, CD47, and the viral glycoprotein. Next, flow virometry is used to calculate the fraction of viral vectors that express both of the exogenous surface proteins. The method is repeated for viral vectors with these exogenous surface proteins produced using the traditional methods. As an alternate to flow virometry, viral vectors are run on a reducing SDS-page gel and stained with secondary antibodies against the exogenous surface proteins to confirm presence in the expected band size.

It is expected that a significant fraction of the viral vectors produced using the methods herein will express both exogenous surface proteins, while double positive expression for viral vectors produced using the traditional production methods employed in the industry will be below the limit of detection.

### Example 6. Viral Vectors Transduce Primary T Cells

In Example 6, the viral vector produced in Example 4 is characterized for the ability to transduce primary human T-cells.

Primary human T cells (Stem Cell Technologies) are thawed and cultured as per manufacturer's protocol in 96 well plates. The viral vector of Example 4 is serially diluted and introduced into each well of the 96 well plate in a titration assay. The primary T cells are assayed by flow cytometry against the GFP transgene and by integration of the transgene into the T cell genome by qPCR. It is expected that the viral vector will be able to transduce T cells across a range of dilutions by both integrating unit and transducing unit assays.

### Example 7. Viral Vectors Transduce T Cells in Mixed Cultures

In Example 7, the viral vector produced in Example 4 is characterized for the ability to transduce the Jurkat T-cells when mixed in culture with other lines.

7 wells of a 96 well plate are seeded in quadruplicate with 10000 cells in various combinations of Jurkat and HEK293 cells. These combinations are: 100% HEK293, 1:1000 Jurkat: HEK293, 1:200 Jurkat: HEK293, 1:100 Jurkat: hek293, 1:20 Jurkat: HEK293, 1:10 Jurkat: HEK293, 100% Jurkat.

Each well is transduced with viral vectors at different dilutions. 3 days later, the cells are analysed by flow cytometry for expression of GFP. It is expected that the frequency of GFP+ cells will correspond to the frequency of jurkat cells in culture.

Furthermore, the vector produced in Example 4 is characterized for the ability to transduce T cells in a population of peripheral blood mononuclear cells (PMBC) that contain both T-Cells and non-T-cells. Human PBMCs are thawed and cultured in 6 well plates. Vector is added to these cultures and incubated for between 12 and 72 hours. Cells are then stained for markers of different cell types and analysed by flow cytometry. Only T cell populations are GFP positive, indicating that the vector only transduces T cells.

### Example 8. Viral Vectors Do Not Transduce Cells That Do Not Express the CD3 Surface Marker

In Example 8, the viral vector produced in Example 4 is characterized for off-target transduction.

A panel of cell lines is cultured according to supplier instructions which represent various cell types in the body. The panel includes the following cell line: HEK293, HEK293 overexpressing CD3, HeLa, Jurkat, hepg2, ac16. These cells are stained using an anti-CD3 antibody and analysed by flow cytometry to confirm expression or lack of expression of CD3. It is confirmed that only HEK293 overexpressing CD3, and Jurkat, lines express CD3.

Next, the panel of cell lines is transduced with dilutions of the viral vector of Example 4 found to be in the linear range in the titration assay, including one dilution above and below the linear range. All cells are analysed by flow cytometry. It is expected that only the CD3 expressing cells will be GFP positive, which would indicate that the viral vector only transduces the cells that express CD3.

### Example 9. Alternate Methods for Chemical Addition of Exogenous Targeting Molecules Onto Vectors

Two additional methods were used to conjugate exogenous targeting molecules onto vectors (Cell type-specific delivery by modular envelope design, Strebinger et al. Nature Communications, 2023). A viral vector core was produced comprising a VSVg glycoprotein or a GP64 glycoprotein. A first handle was introduced into the vector core. In this example, the handle was either an engineered protein comprising the protein AG coding sequence between the secretion and transmembrane domains of VSV-G; or, the handle was a SNAP tag. These vectors were conjugated with either an antibody (using the Fc-domain interaction with the protein AG) or a benzy guanine-modified antibody respectively. These two vector conjugation methods were prepared with anti-CD3, anti-CD5, and anti-CD46 antibodies. The resulting vectors were assayed for transduction capability of Jurkat cells, and efficient gene delivery to the Jurkat cells was observed. A CD117-targeted vector was also produced using these methods and transduction of Kasumi-1 cells was observed.

In this example, several additional vectors were produced with different glycoproteins using the protein AG conjugation system. These glycoproteins include: blinded cocal virus glycoprotein, blinded rhabdovirus glycoprotein, GP64 glycoprotein.

In this example, a vector was also produced where the vector core is a Moloney Murine Leukemia Virus vector (rather than a lentiviral vector), with a SNAP tag conjugated to an anti-CD5 and anti-CD3 antibody. These vector efficiently transduced Jurkat cells (FIG. 5 and supplementary FIG. 5 of Strebinger et al. 2023).

Using the vectors with a blinded VSVG glycoprotein and a SNAP tag handle, conjugated to an anti-CD3, anti-CD28, or anti-CD4 antibody. These vectors efficiently targeted and transduced T-cells in a mixed peripheral blood mononuclear cell culture with GFP, and did not transduce non-T-cells (FIG. 6 of Strebinger et al. 2023).

Next, vectors assembled using the blinded VSVG glycoprotein and a CD5 antibody conjugated using the SNAP tag were produced and injected into mice. Five days post-injection, different cell subsets in the spleen were analysed (Strebinger et al. 2023).

### Example 10. In Vivo Transduction

Wild type mice are injected intravenous with a viral vector preparation or saline. This viral vector is engineered with an exogenous targeting protein targeting T cells and encoding for green fluorescent protein. At one week and two weeks after dosing, blood is drawn from the mouse and analysed by flow cytometry for expression of green fluorescent protein in T cells. Animals are also weighed and examined for any adverse health effects of the viral vector. At two weeks after dosing, the animals are sacrificed and tissue samples from each organ are harvested and analysed for off-target expression of GFP in these tissues.

Mice are also injected with a viral vector preparation engineered with an exogenous targeting protein targeting T cells and encoding for the alkaline phosphatase protein. At one and two weeks after dosing, blood is drawn and analysed by flow cytometry. After two weeks, animals are sacrificed and tissues samples from each organ are harvested and assayed for off-target alkaline phosphatase expression.

These data indicate that this method can be used to express any relevant exogenous functional proteins.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A viral vector core having on its surface a first chemical handle.

2. The viral vector core of claim 1, wherein the first chemical handle comprises an azido sugar.

3. The viral vector core of claim 1, wherein the first chemical handle comprises an alkyne sugar.

4. The viral vector core of any one of claims 1-3, wherein the viral vector core contains a nucleic acid encoding one or more therapeutic proteins.

5. The viral vector core of any one of claims 1-4, wherein the viral vector core is a lentiviral vector core.

6. A composition comprising the viral vector core of any one of claims 1-5.

7. A viral vector comprising one or more exogenous targeting protein(s), one or more exogenous immune modifying protein(s), and/or one or more polymer(s) covalently attached to its surface through a conjugation signature.

8. The viral vector of claim 7, wherein the conjugation signature comprises a triazole bond, a thioether bond, a disulfide bond, or a [4+2] cycloadduct.

9. The viral vector of claim 7 or 8, wherein the one or more exogenous targeting protein(s) or the one or more exogenous immune modifying proteins have a molecular weight of greater than 2 kDa

10. The viral vector of claim 7 or 8, wherein the one or more exogenous targeting proteins comprise an scFv, a nanobody, a darpin, a VHH, an aptamer, or a lectin-binding domain.

11. The viral vector of claim 7 or 8, wherein the polymer is a polyethylene glycol (PEG).

12. The viral vector of claim 11, wherein the PEG has an average molecular weight of about 100 g/mol to about 20000 g/mol.

13. The viral vector of any one of claims 7-12, wherein the viral vector contains a nucleic acid encoding one or more therapeutic proteins.

14. The viral vector of any one of claims 7-13, wherein the viral vector is a lentiviral vector.

15. The viral vector of any one of claims 7-14, wherein the viral vector has a pseudotype selected from the group consisting of: VSV-G, blinded VSV-G, Sindbis glycoprotein, blinded Sindbis glycoprotein, murine leukemia virus glycoprotein, blinded murine leukemia virus glycoprotein, Moloney murine leukemia virus glycoprotein, blinded Moloney murine leukemia virus glycoprotein, cocal virus glycoprotein, blinded cocal virus glycoprotein, foamy virus glycoprotein, blinded foamy virus glycoprotein, and GP64 glycoprotein.

16. The viral vector of any one of claims 7-14, wherein the viral vector does not have a pseudotype of VSV-G.

17. A composition comprising a population of the viral vectors of any one of claims 7-16.

18. The composition of claim 17, wherein the population of the viral vectors has a functional titer that corresponds to greater than about 1E7 TU/mL prior to concentration and conjugation of the viral vector.

19. The composition of claim 17 or 18, wherein the ratio of the functional titre to the physical titre is about 1:1000 to about 1:10.

20. The composition of any one of claims 17-19, wherein the composition is a pharmaceutical composition.

21. A method of making a viral vector, wherein the method comprises:
(a) contacting a viral vector core comprising on its surface a first chemical handle with an exogenous targeting protein, an exogenous immune modifying protein, or a polymer comprising a second chemical handle, and
(b) covalently conjugating the first chemical handle with the second chemical handle, to produce the viral vector.

22. The method of claim 21, wherein the method further comprises:
producing the viral vector core comprising on its surface the first chemical handle.

23. The method of claim 22, wherein the step of producing the viral vector core comprises a step of purifying or isolating the viral vector core by clarification and/or chromatography.

24. The method of claim 22, wherein the step of producing the viral vector core comprises culturing a producer cell in a liquid culture medium comprising a small molecule capable of metabolically inserting the first chemical handle into glycoproteins on the membrane of the producer cell.

25. The method of any one of claims 22-24, wherein the first chemical handle and the second chemical handle are azide and alkyne.

26. The method of any one of claims 22-24, wherein the first chemical handle or the second chemical handle comprise DBCO.

27. The method of any one of claims 22-26, wherein step (b) is performed using a click chemistry reaction.

28. The method of claim 27, wherein the click chemistry reaction is performed at a temperature of about 30° C. to about 40° C., and a pH of about 6.5 to about 7.5.

29. A kit comprising a composition of any one of claims 17-20.

30. A method of treating a subject in need thereof, the method comprising administering to the subject a therapeutically effective amount of a composition of any one of claims 17-20.
